(19) United States
(12) Patent Application Publication
(10) Pub. No.: US 2008/0200819 A1
(43) Pub. Date: Aug. 21, 2008

(54) Orthostasis detection system and method

(76) Inventors: Lawrence A. Lynn, Columbus, OH (US); Donald Lorry Freidenberg, Upper Arlington, OH (US)

Correspondence Address: NELLCOR PURITAN BENNETT LLC;ATTN: IP LEGAL, 60 Middletown Avenue, North Haven, CT, 06473, US

(21) Appl. No.: 11/708,314

(22) Filed: Feb. 20, 2007

(51) Int. Cl. A61B 5/021

(52) U.S. Cl. 600/485

(57) ABSTRACT

The disclosed embodiments relate to a system and method for monitoring patient data. An exemplary method comprises obtaining hemodynamic variation data that corresponds to a variation in intravascular hemodynamics of a patient, searching the hemodynamic variation data for an indication of orthostasis in response to the occurrence of a positional maneuver of or by the patient, and generating an output if the indication orthostasis is discovered.

### FIELD OF THE INVENTION

This invention relates systems and methods for detecting and monitoring adverse disorders in clinical medicine.

### BACKGROUND

Acute reductions in venous return and, in particular, orthostasis resulting from positional induction of acute reduction of venous return are potential problems in hospitals, nursing homes and in the home environment. Orthostasis, which is a sudden fall in blood pressure when a person assumes a standing position, is a cause of falls and injury and is commonly induced by medication. The conventional standard technique for detecting orthostasis using paired supine and standing blood pressure measurements is cumbersome and, therefore, often not applied by medical personnel, placing the patient at risk for repeated falls. Also, the conventional technique provides only a limited picture of the hemodynamic response to position change. The blood pressure may drop suddenly and quickly return so that the standing blood pressure may miss the drop. The standing or the Valsalva maneuvers are both associated with both a fall in venous return (which reduces stroke volume) and compensatory vasoconstriction. Both of these physiologic events cause a fall in the waveform amplitude of a plethysmographic pulse signal of a patient in response to the maneuver. In some patients with autonomic dysfunction, compensatory vasoconstriction is defective; however, these patients may have severe drops in venous return so that a fall in the pleth waveform amplitude still occurs with standing. An improved system and method of detecting orthostasis is desirable.

### BRIEF DESCRIPTION OF THE DRAWINGS

FIG. 1 is a block diagram of a system that is adapted to analyze data corresponding to variations in a plethysmographic pulse signal in accordance with an exemplary embodiment of the present invention; and

FIG. 2 is a process flow diagram illustrating a method of processing patient data in accordance with an exemplary embodiment of the present invention.

### DETAILED DESCRIPTION

An exemplary embodiment of the present invention comprises an orthostasis detection system and method and a venous return assessment system and method. Furthermore, exemplary embodiments of the present invention may comprise a system and method to identify a timed pattern of orthostasis to, for example, identify patients with more sustained patterns of blood pressure fall or with incomplete recovery after the fall. Accordingly, an exemplary orthostasis detection system comprises a hemodynamic signal detector, such as a pulse oximeter, an input device for automatically or manually inputting an occurrence of a maneuver, such as standing or a Valsalva maneuver), and a processor for generating a time series of a hemodynamic signal (such as a plethysmographic pulse signal) and for outputting an indication based on both the maneuver and the time series. In one exemplary embodiment, the processor is programmed to determine at least one variation of the pulse signal (such as the systolic variation of the plethysmographic pulse), to output a time series of the variation and to detect a threshold and/or pattern of variation and to output an indication based on the detection. The variation of the plethysmographic pulse signal is one example of hemodynamic variation data that corresponds to a variation in intravascular hemodynamics of a patient. In another exemplary embodiment, the processor outputs a signal corresponding to at least one pleth waveform component prior to standing (such as the amplitude of the pleth signal, for example, the average minimum of the pleth signal, the average maximum amplitude of the pleth signal, or a value indicative of a respiratory-related plethysmographic waveform variation). The processor then outputs the pattern or value indicative of at least one pleth waveform component after standing and then compares the value or pattern prior to standing with the value or pattern after standing. Examples of input devices that can be used to detect when the patient stands include, for example, a patient-mounted position sensor or a manual input device such as a mouse or keyboard. In an exemplary embodiment of the present invention, a position sensor is worn by a patient to communicate postural position information about the patient to the processor. The processor can determine and/or calculate the difference between the pre-standing and post standing values. Exemplary embodiments of the present invention may provide an orthostasis detection system that is simple, inexpensive, non-invasive, automated, and can be employed in a long-term ambulatory state.

One exemplary embodiment of detecting orthostasis according to the present invention comprises measuring at least one pleth waveform component, inputting the occurrence of a maneuver on a patient into a processor, measuring at least one pleth waveform component after the maneuver, comparing the pleth waveform component measured before the maneuver to the pleth waveform component after the maneuver. Another exemplary embodiment includes the acts of deriving a time series of a pleth waveform component, providing an indication of the time of at least one maneuver along the time series and outputting the time series. Another exemplary embodiment may include the act of comparing a pleth waveform pattern before a maneuver to the pleth waveform pattern after the maneuver.

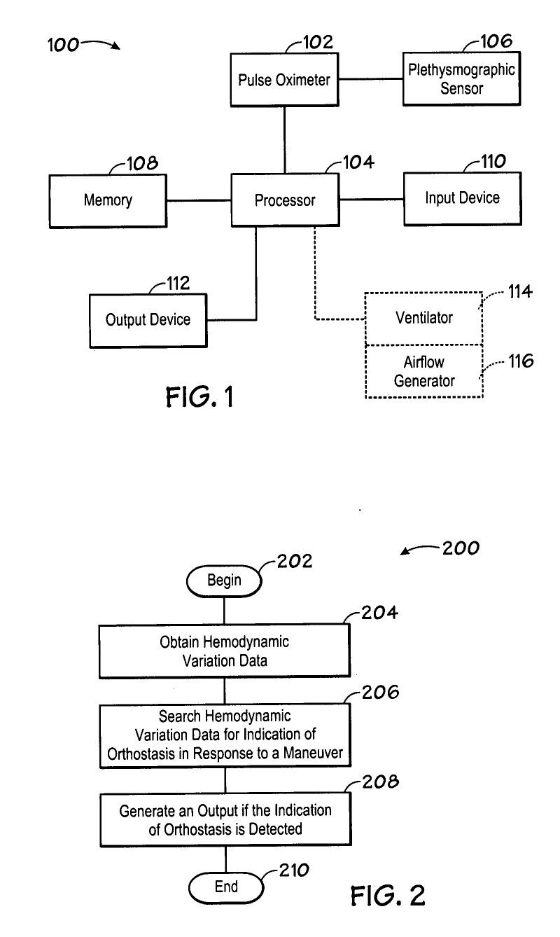

FIG. 1 is a block diagram of a system that is adapted to analyze data corresponding to variations in a plethysmographic pulse signal in accordance with an exemplary embodiment of the present invention. The system is generally referred to by the reference number 100. The system 100 comprises a pulse oximeter 102, which is connected to a processor 104. The processor 104 may be programmed to perform calculations and analysis on data corresponding to variations in a plethysmographic pulse signal. In the exemplary embodiment illustrated in FIG. 1, the pulse oximeter 102 is adapted to receive plethysmographic pulse data from a plethysmographic sensor 106, which may be connected to a patient. In an alternative embodiment, the processor 104 may be adapted to analyze previously obtained data stored in a memory 108, which is coupled to the processor 104. The exemplary system 100 may include an input device 110 to signal the performance of a maneuver by or on a patient. In this way, data being evaluated by the system 100 may be analyzed in the context of when it occurred relative to the performance of the maneuver. While an exemplary embodiment of the invention comprises the pulse oximeter 102. other devices that detect and/or monitor a hemodynamic pulse related parameter such as, for example, a pressure transduced arterial catheter, a continuous blood pressure monitor, or a digital volumetric plethysmograph, to name a few, may be employed to detect the hemodynamic and systolic pressure variations discussed below. The system 100 may additionally include an output device 112, such as a printer, display device, alarm or the like. The output device 112 may be adapted to signal or provide an indication of a condition detected by the processor 104.

Those of ordinary skill in the art appreciate that the detection and quantification of at least one pleth waveform component (such as magnitude of the respiratory related variation of the pleth) is possible. One method of processing the pleth signal is described in U.S. Pat. No. 7,081,095 (the contents of which are incorporated by reference as if completely disclosed herein). An example of a pleth waveform component is the pleth variation associated with ventilation as calculated from the plethysmographic pulse of the pulse oximeter 102, which is a sensitive indicator of intravascular blood volume in patients undergoing mechanical ventilation. The plethysmographic waveform (or pulse) variation can, for example, be outputted as a percentage of the peak pleth amplitude (see, for example, Pulse Oximetry Plethysmographic Waveform During Changes in Blood Volume, British Journal of Anesthesia, 82 (2): 178-81 (1999), the contents of which are hereby incorporated by reference as if completely disclosed herein).

However, while a decrease in effective venous return (as induced by a decrease in blood volume) commonly increases the respiratory-related pleth waveform (or systolic pressure) variation, a rise in respiratory effort can also increase this variation so that the linkage of this variation to the intravascular volume becomes much more complex in spontaneously breathing patients. Simplistic approaches, which attempt to determine the trend of the this plethysmographic waveform variation to determine blood volume, can provide a false trend which may suggest a falling blood volume due to a plethysmographic waveform variation cased by a rising respiratory effort due to bronchospasm, pulmonary embolism, or even an excess in blood volume inducing pulmonary edema.

The inventors of the present invention has recognized that, because the pleth waveform variation increases with both a fall in effective venous return or an increase in respiratory effort (which can be associated with excess venous return and heart failure and increases in lung water), the pattern of the pleth waveform variation (or other pleth waveform components) are best analyzed in timed relation to a maneuver (such as a standing maneuver), which is known to reduce venous return, especially in disease states and in the presence of certain medications or in states of low blood volume so that the relationship of the change in pleth waveform variation to the maneuver can be determined to thereby better establish the presence of reduced venous return and to identify when the magnitude of venous return and/or the vasoconstrictive arterial response to a decline in venous return, is abnormal.

In an exemplary embodiment of the present invention, the processor 104 is programmed to detect a falling SPO2 combined with a rising magnitude of the pleth respiratory variation or a change or a pattern of change in a plethysmographic pulse component in relation to a maneuver that potentially reduces venous return, such as standing. In an exemplary embodiment of the present invention, the processor 104 can be programmed, as by using an objectification method, to convert the plethysmographic time series into program objects such as dipoles (see, e.g. U.S. patent application Ser. No. 10/150,842 filed on Aug. 21, 2003 (now U.S. Patent Publication No. 20030158466), the contents of which are incorporated by reference as if completely disclosed herein) and objects comprised of events such as rises and falls and reciprocations (fundamental level).

Reciprocation objects can be defined by the user or by adaptive processing, as a threshold or pattern of reduction of amplitude, peak value, nadir value, slope, area under the curve (AUC) or the like. The components of the rises and falls such as the peaks, the nadirs, the slopes, or the AUC, to name a few, can be applied to render the composite level of the plethysmographic time series. The pattern of the reciprocations of one or more of these values (the composite level) can use used to detect respiration rate wherein the respiration rate is defined as the average number of reciprocations at the composite level per minute. More complex variations in the pattern of the plethysmographic pulse will also be detectable at the composite level such as apneas or sustained variations in blood flow to the finger (as, for example, may be induced by a mechanical ventilator setting change or a change in body position from the supine to the upright position). The SPO2 can be similarly processed in parallel with the pulse and the pattern of the pulse at the any level of the pulse compared with the pattern of the SPO2 at any level.

In an exemplary embodiment of the present invention, the number of reciprocations per minute and/or the magnitude of the amplitude of the reciprocations, amplitude, as determined by calculating the number of reciprocations per minute, is compared using the processor 104 with the time series of the SPO2 at, for example, the raw, dipole or fundamental level. The relationship between these two time series determined by the processor 104 may be used to detect and quantify the relationship between the ventilation time series (derived of the plethysmographic pulse) and the oxygen saturation time series.

In an exemplary embodiment of the present invention, the processor 104 is programmed to detect a change (such as a fall) in a plethysmographic pulse component (as for example the components noted above) in response to a maneuver, which affects venous return to the heart. Examples of such maneuvers include changes in a mechanical ventilator (such as an increase in positive pressure delivery to the patient, an increase in positive and expiratory pressure delivery to the patient, a change or changes in tidal volume, PEEP, respiration rate, or I:E ratio). Other maneuvers can include having the patient stand up from a supine position to detect a positional variation in the plethysmographic pulse parameter. The processor 104 can be programmed to automatically detect the maneuver or to receive an input from the input device 110 indicative of the occurrence or pattern of the maneuver. In an exemplary embodiment of the present invention, the input device 110 can be accessed through a menu which can allow the user to specify the maneuver (such as standing or exercising).

In an exemplary embodiment of the present invention, the processor 104 is adapted to detect orthostasis. An input is provided via the input device 110 when the patient undergoes a maneuver such as a change in body position (for example, standing up). The beginning of the maneuver may be taken into account when analyzing the corresponding SPO2, respiration and ventilation data. A variation in a least one component of the plethysmographic pulse may be quantified and a relationship between the variation and the maneuver may be identified. By way of example, a fall in the average pleth amplitude (such as the systolic variation ) of about 20% or more in response to standing can result in an output that indicates to an attendant that there is a need to check the blood pressure in the supine and standing position. Alternatively, the processor 104 can be programmed to detect an increase in the reciprocation amplitude at the composite level of about 20-40% or more can output an indication of the presence and/or magnitude and/or pattern of orthostatic variation in the pleth amplitude pattern. In one exemplary embodiment of the present invention, the pulse oximeter 102 is adapted to be used for spot checks of the SPO2. The system may also be adapted to display a menu on, for example, either the input device 110 or the output device 112 depending on system design considerations. A user may specify that one or more orthostatic variation maneuver(s) is (are) to be initiated via the menu. The user may then be instructed to press a button or touch the screen at the time the patient stands up. The processor 104 tracks the pattern of the pleth and outputs and detects threshold pattern changes or lack thereof as noted above. An indication (such as a textual indication or alarm) of the presence or absence of threshold orthostatic variation value and/or pattern may be provided. In addition, the slope or other components of the pattern of the variation subsequent to the maneuver can be determined and quantified. A time series indicative of the variation with the points of the standing or other position change marked along the time series may be outputted for over reading by the physician. Furthermore, a time series of one or more of the maneuvers may also be created. A time series of pleth variation data may be compared to the time series of one or more maneuvers.

In an alternative embodiment of the invention, the input device 110 comprises a position sensor adapted to be mounted in connection and/or in communication with one or more components of the system 100. The position sensor, which may be mounted to the chest of a patient during sleep studies, can alternatively be configured for mounting on the thigh of the patient so that detection of the change form recumbent to standing or sitting to standing is automatically performed by the position sensor. The plethysmographic monitor system 100 can have memory such as the memory 108 in wired and/or wireless communication therewith to allow ambulatory detection and quantification of the time series pattern of at least one plethysmographic pulse variation (such as a time series of the systolic pleth variation) in relation to variations in body position over a time interval such as 8-24 hours or more. Moreover, hemodynamic variation data may be obtained by ambulating a patient who is wearing a mobile hemodynamic variation detector. One example of ambulating the patient includes standing the patient while the patient is wearing a mobile hemodynamic variation detector. An exemplary embodiment of the present invention provides an ambulatory orthostasis detection system useful for titration of medications known to cause orthostasis. For example, the orthostasis monitor may be wrist mounted with the probe extending to the index finger. Such systems can be useful for the titration of medications used for the treatment of dementia in nursing homes or in the home environment. The system can also be used to titrate medication used for the treatment of heart failure or hypertension and for the ambulatory investigation of the cause of syncope or lightheadedness.

In an exemplary embodiment of the present invention, the processor 104 is programmed to compare a time series of body position to a parallel time series of at least one component of the pleth variation. The time series of at least one component of the pleth variation is analyzed to detect patterns, which occur in relation to changes in body position. In addition, the pattern of the heart rate can be identified in relation to body position so that heart rate acceleration can be detected and quantified for example.

In another exemplary embodiment of the present invention, the plethysmographic monitor system 100 serves as a pulse rate and pattern detection system. The processor 104 is programmed to determine the time intervals of the pleth including the time between pulses, and the time of systole, the time of diastole, the time of the rise, the time of the fall, and the pattern of pulses. Different patterns can be detected such as the pattern of atrial fibrillation (for example, identified by detecting an irregularly irregular interval between pulses and/or an irregularly irregular pulse amplitude), or a paroxysmal tachycardia (for example, detected by noting a precipitous increase in pulse rate which resolves precipitously). This pulse rhythm and pulse amplitude diagnostic function is complementary to the orthostasis detection function for the evaluation of lightheadedness and syncope.

In yet another exemplary embodiment of the present invention, a time series of the respiratory rate (as for example determined from the pleth), a time series of the pleth variation, and a time series of the SPO2 are compared to identify the pattern relationships between these parameters such as a rise in pleth variation and a fall in SPO2, a rise in pleth variation and rise in respiratory rate, and/or a rise in respiratory rate and a fall in SPO2 and /or in relation to a maneuver such as standing. The processor 104 may be programmed to detect pathophysiologic divergence of the respiratory rate and/or the pleth variation and/or the SPO2.

In an exemplary embodiment of the present invention, an associated processor may be programmed to detect an oxygen saturation parameter (such as the ratio of ratios and/or the SPO2) and a respiration parameter (such as the respiration rate) and a magnitude of pleth variation. For example, the magnitude of pleth variation may be determined by the pleth amplitude and/or pleth slope variation. The pattern of the time series of the respiratory rate may then be compared with the pattern of the SPO2 to detect and abnormal relationship, such as pathophysiologic divergence with an increasing difference between the respiratory rate and the SPO2, for example. The processor may be programmed to output an indication based on the detection of the pattern or absolute value of the relationship and/or to output an index value indicative the relationship. The detection of a rise in respiration rate associated with a fall in plethysmographic pulse variation can be detected, quantified, and the pattern of the relationship analyzed and tracked by the processor. The processor can be programmed to provide an updated indication of the relationship and the pattern of the relationship to the user. The method of processing can, for example, be of the type discussed in U.S. Pat. No. 7,081,095 (the contents of which is incorporated by reference as if completely disclosed herein). In an exemplary embodiment of the present invention, a plurality of parameters are combined to determine the global respiratory variation, including the amplitude of the events (at the fundamental level), the variation of the peak values (fundamental level), and the variation of the nadirs (also fundamental level).

The system 100 may comprise an optional ventilator 114 operatively coupled to the processor 104. The ventilator 114 may comprise an airflow generator 116 that is adapted to an airflow to a patient. The processor 104 may be programmed so that the time series of the systolic pleth variation (for example) is displayed on the output device 112 adjacent a time series of at least one ventilation parameter. The processor 104 can be programmed for example to detect a pattern or threshold increase in systolic pressure variation in relation to a ventilator change and to output an indication of the pattern or threshold increase to the operator.

FIG. 2 is a process flow diagram illustrating a method of processing patient data in accordance with an exemplary embodiment of the present invention. The diagram is generally referred to by the reference number 200. At block 202, the process begins.

At block 204, hemodynamic variation data is obtained. The hemodynamic variation data, which corresponds to a variation in intravascular hemodynamics of a patient, may be obtained, for example, from a memory device or directly from monitoring a patient in real time. At block 206, the hemodynamic variation data is searched for an indication of orthostasis in response to a maneuver performed on or by the patient. An output, such as an alarm, printout and/or display, is generated if the indication of orthostasis is detected, as indicated at block 208. At block 210, the process ends.

One exemplary embodiment of the present invention comprises a method for determining the proper dose of a medication comprising administering the medication, monitoring the patient for orthostasis using the aforementioned methods, adjusting the dose of the medication based on the monitoring. The medications can include, for example, tricyclic antidepressants, MOAI, atypical antipsychotic agents, dopamine agonists, Flomax, Hytrin, diuretics, calcium channel blockers, and ACE inhibitors to name a few. Another exemplary embodiment comprises monitoring the patients with diseases or disorders for orthostasis using the aforementioned methods. The diseases or disorders can include, for example, dementia, Parkinsonian syndromes, diabetic neuropathy, and/or POTS to name a few.

In one exemplary embodiment of the present invention, an automated BP device is modified to perform automated orthostasis evaluation on demand. The processor of the device is programmed to first measure the blood pressure, automatically or manually receive an input of a maneuver, such as a standing maneuver, then automatically acquire consecutive blood pressure readings in rapid sequence to determine and record a time series of blood pressure readings after the maneuver and to output an indication based on the time series. The automatic blood pressure device can be programmed to display a menu offering the operator an automated orthostasis evaluation. The processor can be programmed to constrict the cuff on the second measurement to a point to a lower pressure point than the systolic pressure (such as the point wherein the diastolic pressure had been identified on the first measurement) and then to record and analyze the pulse tracing detected under the partially constricted cuff (as by the aforementioned methods) for pulse variations during and after a maneuver. In one exemplary embodiment, the automated BP cuffs provide a selection in its display for orthostasis evaluation so the orthostasis can become a standard vital sign for some patient populations.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A system that is adapted to monitor patient data, comprising:
a plethysomographic sensor adapted to obtain hemodynamic variation data that corresponds to a variation in intravascular hemodynamics of a patient;
a processor that is adapted to search the hemodynamic variation data for an indication of orthostasis in response to the occurrence of a positional maneuver of or by the patient; and
an output device that is adapted to generate an output if the indication orthostasis is discovered.

2. The system recited in claim 1, comprising a mobile hemodynamic variation detector disposed on the patient, the mobile hemodynamic variation detector being adapted to deliver the hemodynamic variation data to the processor.

3. The system recited in claim 1, wherein the processor is adapted to detect an occurrence of the positional maneuver.

4. The system recited in claim 1, comprising a mobile hemodynamic variation detector disposed on the patient, the mobile hemodynamic variation detector being adapted to deliver the hemodynamic variation data to the processor when the positional maneuver comprises moving from a supine position to a standing position.

5. The system recited in claim 1, comprising a position sensor that is adapted to provide data corresponding to a postural position of the patient.

6. The system recited in claim 1, wherein the processor is adapted to generate a time series of the hemodynamic variation data.

7. The system recited in claim 6, wherein the processor is adapted to:
detect an occurrence of the positional maneuver; and
detect along the time series an indication of orthostasis in association with the occurrence of the positional maneuver.

8. The system recited in claim 7, wherein the processor is adapted to:
detect an occurrence of the positional maneuver; and
detect along the time series an indication of orthostasis subsequent to the occurrence of the positional maneuver.

9. The system recited in claim 1, wherein the processor is adapted to:
detect a plurality of sequential positional maneuvers; and
detect along the time series an indication of orthostasis in association with at least one of the plurality of sequential positional maneuvers.

10. The system recited in claim 1, wherein the processor is adapted to receive an input indicative of the occurrence of the positional maneuver.

11. The system recited in claim 1, wherein the positional maneuver comprises a standing maneuver.

12. A system for monitoring patient data, comprising:
means for obtaining hemodynamic variation data that corresponds to a variation in intravascular hemodynamics of a patient;
means for searching the hemodynamic variation data for an indication of orthostasis in response to the occurrence of a positional maneuver of or by the patient; and
means for generating an output if the indication orthostasis is discovered.

13. A tangible machine-readable medium, comprising:
code adapted to obtain hemodynamic variation data that corresponds to a variation in intravascular hemodynamics of a patient;
code adapted to search the hemodynamic variation data for an indication of orthostasis in response to the occurrence of a positional maneuver of or by the patient; and
code adapted to generate an output if the indication orthostasis is discovered.

14. The tangible medium recited in claim 13, comprising code adapted to detect an occurrence of the positional maneuver.

15. The tangible medium recited in claim 13, comprising code adapted to generate a time series of the hemodynamic variation data.

16. The tangible medium recited in claim 13, comprising:
code adapted to detect an occurrence of the positional maneuver; and
code adapted to detect along the time series an indication of orthostasis in association with the occurrence of the positional maneuver.

17. The tangible medium recited in claim 13, comprising:
code adapted to detect an occurrence of the positional maneuver; and
code adapted to detect along the time series an indication of orthostasis subsequent to the occurrence of the positional maneuver.

18. The tangible medium recited in claim 13, comprising:
code adapted to detect a plurality of sequential positional maneuvers; and
code adapted to detect along the time series an indication of orthostasis in association with at least one of the plurality of sequential positional maneuvers.

19. The tangible medium recited in claim 13, comprising code adapted to receive an input indicative of the occurrence of the positional maneuver.
